(19) United States
(12) Patent Application Publication
(10) Pub. No.: US 2021/0052544 A1
(43) Pub. Date: Feb. 25, 2021

(54) METHODS OF TREATING AND/OR PREVENTING BEDSORES USING NABILONE

(71) Applicant: Solantech Inc., Newmarket (CA)

(72) Inventor: Shabbir AMANULLAH, London (CA)

(21) Appl. No.: 16/965,483

(22) PCT Filed: Jan. 21, 2019
(86) PCT No.: PCT/CA2019/050072

§ 371 Date: Jul. 28, 2020

Related U.S. Application Data
(60) Provisional application No. 62/623,173, filed on Jan. 29, 2018.

(51) Int. Cl. A61K 31/352; A61L 15/44; A61K 9/00; A61K 45/06; A61P 17/02

(52) U.S. Cl. CPC A61K 31/352; A61L 15/44; A61L 2300/402; A61K 45/06; A61P 17/02; A61K 9/0053

(57) ABSTRACT

Compositions for treating bedsores and methods for treating bedsores are provided. The compositions include a therapeutically effective amount of nabilone and may be formulated for oral or topical administration. The composition may further include a second therapeutic agent such as antifungal agent, steroid, antibiotic, analgesic, and combinations thereof.

### FIELD OF THE INVENTION

This invention pertains generally to treatment of skin disorders and in particular, to methods of treating and/or preventing bedsores using nabilone.

### BACKGROUND OF THE INVENTION

Bedsores, also called pressure sores or decubitus ulcers, are localized sores or damage to the skin and underlying tissue that result from prolonged periods of pressure on the skin. Bedsores are most prevalent on skin over bony prominences including sacrum, coccyx, heel and hip. The back of the head, the elbows, ankles and shoulders are also common sites.

Limited mobility and ability to change position significantly increase the risk of bedsores. Bedsores affect over 2.5 million patients annually in the U.S. and there is a direct correlation between hospital bedsores and patient mortality rate. In particular, hospital-acquired bedsores is a significant risk factor associated with mortality. In Canada, patients with bedsores tend to stay four days longer in hospitals, are 7% more likely to die and on average cost the health care system an additional $13,500. The annual cost associate with management and treatment of bedsores is significant. In addition, hospital-acquired bedsores are commonly claimed in wrongful death suits.

Effective prevention and treatment of bedsores would positively impact patient quality of life and reduce health care expenses.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide methods of treating and/or preventing bedsores using nabilone. In accordance with an aspect of the present invention, there is provided a method of treating bedsores comprising administering to a patient an effective amount of nabilone, optionally the nabilone is administered orally or topically.

In accordance with another aspect of the invention there is provided a composition for treating bedsores comprising a therapeutically effective amount of nabilone, optionally the composition is formulated for oral administration or topical administration.

In accordance with some embodiments, the composition is formulated as a gel, salve, foam or cream for direct application to the bedsore.

In accordance with another aspect of the invention there is provided a composition for treating bedsores comprising a therapeutically effective amount of nabilone and a second therapeutic, optionally the second therapeutic is selected from the group consisting of antifungal agent, steroid, antibiotic, analgesic, and combinations thereof.

In accordance with another aspect of the invention there is provided a wound dressing for treating bedsores comprising a wound dressing and a composition comprising a therapeutically effective amount of nabilone.

### DETAILED DESCRIPTION OF THE INVENTION

Synthetic cannabindiols such as nabilone are provided for the administration orally or topically for the treatment and/or prevention of bedsores.

Compositions for treatment and/or prevention of bedsores include nabilone as an active agent and may include additional therapeutic agents and/or carriers. Additional therapeutic agents include antifungal agents, steroids, antibiotics, analgesics, and combinations thereof. In some embodiments, the topical composition may also include components to facilitate absorption and/or carriers.

To gain a better understanding of the invention described herein, the following examples are set forth. It will be understood that these examples are intended to describe illustrative embodiments of the invention and are not intended to limit the scope of the invention in any way.

### EXAMPLES

Patients with dementia having agitation were treated orally at a dose of 0.5 mg nabilone twice a day initially and then going up to a total of 2 mg per day to reduce agitation. A positive clinical side effect of this treatment was that there was a perceived reduction in the number and/or severity of bedsores in those who we were treated. It was further observed that in those patients who did have bedsores, the bedsores appeared to heal faster. The number of patients where this effect was observed was six. The perceived impact on healing was significant with healing times varying between 3-6 weeks for small sores. No large sores were observed in treated patients.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

We claim:

1. A method of treating bedsores comprising administering to a patient an effective amount of nabilone.

2. The method of claim 1, wherein the nabilone is administered orally.

3. The method of claim 1, wherein the nabilone is administered topically.

4. A composition for treating bedsores comprising a therapeutically effective amount of nabilone.

5. The composition of claim 4, wherein the composition is formulated for oral administration.

6. The composition of claim 4, wherein the composition is formulated for topical administration.

7. The composition of claim 6, wherein the composition is formulated as a gel, salve, foam or cream.

8. The composition of claim 6 or 7, wherein the composition comprises a second therapeutic agent.

9. The composition of claim 8, wherein the second therapeutic is selected from the group consisting of antifungal agent, steroid, antibiotic, analgesic, and combinations thereof.

10. A wound dressing for treatment of bedsores comprising a wound dressing and the composition of any one of claims 4 to 9.
